(19) United States
(12) Patent Application Publication
(10) Pub. No.: US 2024/0423983 A1
(43) Pub. Date: Dec. 26, 2024

(54) ENHANCEMENT OF CD47 BLOCKADE THERAPY WITH DHFR INHIBITORS

(71) Applicant: PF Argentum IP Holdings LLC, New York, NY (US)

(72) Inventors: Robert Adam UGER, Richmond Hill (CA); Gloria Hoi Ying LIN, Oakville (CA)

(73) Assignee: PF Argentum IP Holdings LLC, New York, NY (US)

(21) Appl. No.: 18/557,381

(22) PCT Filed: Apr. 25, 2022
(86) PCT No.: PCT/IB2022/053827

§ 371 Date: Oct. 26, 2023

Related U.S. Application Data
(60) Provisional application No. 63/253,125, filed on Oct. 6, 2021.
(60) Provisional application No. 63/180,604, filed on Apr. 27, 2021.

(51) Int. Cl. A61K 31/519; A61K 31/714; A61K 38/00; C07K 14/705

(52) U.S. Cl. CPC A61K 31/519; A61K 31/714; C07K 14/70503; A61K 38/00; C07K 2319/30

(57) ABSTRACT

Materials and methods useful for therapy, including cancer therapy, that combine an agent that blocks the CD47/SIRPα interaction with a DHFR inhibitor are provided.

### FIELD OF THE INVENTION

This invention relates to methods of using an agent that blocks the CD47/SIRPα interaction. More particularly, the invention relates to methods and means that, in combination, are useful for improving cancer therapy.

### BACKGROUND TO THE INVENTION

Cancer cells are targeted for destruction by antibodies that bind to cancer cell antigens, and through recruitment and activation of macrophages by way of Fc receptor binding to the Fc portion of that antibody. Binding between CD47 on cancer cells and SIRPα on macrophages transmits a “don't eat me” signal that enables many tumour cells to escape destruction by macrophages. It has been shown that inhibition of the CD47/SIRPα interaction (CD47 blockade) will allow macrophages to “see” and destroy the target CD47+ cancer cell. The use of SIRPα to treat cancer by CD47 blockade is described in WO 2010/130053, incorporated herein by reference.

International Patent Application Publication No. WO 2014/094122, incorporated by reference in its entirety, describes a protein drug that inhibits the interaction between CD47 and SIRPα. This CD47 blockade drug is a form of human SIRPα that incorporates a particular region of its extracellular domain linked with a particularly useful form of an IgG-based Fc region. In this form, the SIRPαFc drug shows dramatic effects on the viability of cancer cells that present with a CD47+ phenotype. The effect is seen particularly on acute myelogenous leukemia (AML) cells, and on many other types of cancer. A soluble form of SIRP having significantly altered primary structure and enhanced CD47 binding affinity is described in WO 2013/109752, incorporated herein by reference in its entirety.

Other CD47 blockade drugs have been described in the literature and these include various CD47 antibodies (see for instance Stanford's U.S. Pat. No. 8,562,997, and InhibRx′ WO2014/123580), each comprising different antigen binding sites but having, in common, the ability to compete with endogenous SIRPα for binding to CD47, thereby to allow interaction with macrophages and, ultimately, to increase the rate of CD47+ cancer cell depletion. These CD47 antibodies have activities in vivo that are quite different from those intrinsic to SIRPα-based drugs. The latter, for instance, display negligible binding to red blood cells whereas the opposite property in CD47 antibodies creates a need for strategies that accommodate the drug “sink” that follows administration.

Still other agents are proposed for use in blocking the CD47/SIRPα axis. These include CD47Fc proteins (see Viral Logic's WO2010/083253), and SIRPα antibodies as described in UHN's WO2013/056352, Stanford's WO2016/022971, Eberhard's U.S. Pat. No. 6,913,894, and elsewhere.

The CD47 blockade approach in anti-cancer drug development shows great promise. It would be useful to provide methods and means for improving the effect of these drugs, and in particular for improving the effect of the CD47 blockade drug forms, especially those that incorporate SIRPα.

### SUMMARY OF THE INVENTION

It is now shown that the anti-cancer effect of CD47 blockade is improved when combined with a dihydrofolate reductase inhibitor (DHFRi), or anti-folate, such as pralatrexate. More particularly, significant improvement in cancer cell depletion is seen when CD47⁺ cancer cells are treated with a CD47 blocking agent (also referred to herein as a CD47 blockade drug), such as a SIRPα-based drug, in combination with a DHFRi. The two drugs synergize in their effects on cancer cells, and result in the depletion of more cancer cells than can be accounted for by the sum of their individual effects, i.e., with background subtracted, the % phagocytosis of the combination is greater than the added % phagocytosis from SIRPaFc and pralatrexate separately.

In one aspect, there is provided a method for treating a subject presenting with CD47+ cancer cells, comprising administering a treatment-effective drug combination comprising a CD47-binding form of SIRPα or another form of anti-CD47 agent, and a DHFRi, such as pralatrexate.

In a related aspect, there is provided the use of a SIRPα-based drug in combination with a DHFRi for the treatment of a subject presenting with CD47+ cancer.

There is also provided, in another aspect, a combination of anti-cancer agents, i.e., drugs, comprising a CD47 blockade drug such as a soluble SIRPα-based drug (or another form of anti-CD47 agent) and a DHFRi, together with instructions teaching their use in the treatment method herein described.

To the extent that embodiments, details, or variations are described herein with reference to one particular SIRPα-based drug or DHFRi, it should be understood that the same embodiments, details, and variations are intended to apply to others identified herein, unless the application or context explicitly indicates otherwise.

Various details and aspects are described herein as treating or methods of treating. In all such circumstances, it should be understood that related or equivalent aspects include the peptides, analogs, derivatives, or compositions described herein for use in treatment; and the peptides, analogs, derivatives, or compositions described herein for use in the manufacture of medicaments for treatment of diseases or conditions described herein.

The headings herein are for the convenience of the reader and not intended to be limiting. Other aspects of the invention will be apparent from the detailed description and claims that follow.

Exemplary embodiments (E) of the invention provided herein include:
E1. A method for treating a subject presenting with CD47⁺ cancer cells, comprising administering to the subject a CD47 blocking agent/blockade drug, and a dihydrofolate reductase inhibitor (DHFRi).
E2. A method for improving the treatment of a subject presenting with CD47⁺ cancer cells, said subject being treated with a CD47 blocking agent, the method comprising administering to the subject a dihydrofolate reductase inhibitor (DHFRi).
E3. A method for improving the treatment of a subject presenting with CD47⁺ cancer cells, said subject being treated with a dihydrofolate reductase inhibitor (DHFRi), the method comprising administering to the subject a CD47 blocking agent.
E4. A CD47 blocking agent and a dihydrofolate reductase inhibitor (DHFRi) for use in combination to treat a subject presenting with CD47⁺ cancer cells.
E5. A dihydrofolate reductase inhibitor (DHFRi) for use in combination with a CD47 blocking agent to treat a subject presenting with CD47⁺ cancer cells.
E6. Use of a CD47 blocking agent in the manufacture of a medicament for use in combination with a dihydrofolate reductase inhibitor (DHFRi) for the treatment of cancer in a subject presenting with CD47+ cancer cells.
E7. Use of a dihydrofolate reductase inhibitor (DHFRi) in the manufacture of a medicament for use in combination with a CD47 blocking agent for the treatment of cancer in a subject presenting with CD47+ cancer cells.
E8. The method, use, or drug-for-use according to any one of embodiments 1-7, wherein the DHFRi comprises methotrexate or pralatrexate.
E9. The method, use, or drug-for-use according to any one of embodiments 1-7, wherein the DHFRi comprises pralatrexate.
E10. The method, use, or drug-for-use according to any one of embodiments 1-9, wherein the CD47 blocking agent comprises a CD47-binding form of human SIRPα.
E11. The method, use, or drug-for-use according to embodiment 10, wherein the CD47-binding form of human SIRPα is a CD47-binding fragment of human SIRPα.
E12. The method, use, or drug-for-use according to embodiment 11, wherein the CD47 binding fragment of human SIRPα comprises the V region of human SIRPα.
E13. The method, use, or drug-for-use according to any one of embodiments 1-12, wherein the CD47 blocking agent comprises an Fc fusion protein comprising the V region of soluble human SIRPα variant 2 attached to an antibody constant region (Fc).
E14. The method, use, or drug-for-use according to embodiment 13, wherein the Fc fusion protein comprising soluble SIRPα comprises SEQ ID NO: 9 or SEQ ID NO: 10.
E15. The method, use, or drug-for-use according to any one of embodiments 1-9, wherein the CD47 blocking agent comprises soluble SIRPα having one or more amino acid substitutions selected from. L4V/I, V6I/L, A21V, V27I/L, 131T/S/F, Q37W/H, E47V/L, K53R, E54Q/P, H56P/R, S66T/G, K68R, V92I, F94V/L, V63I, M72R, and F103V
E16. The method, use, or drug-for-use according to any one of embodiments 1-9, wherein the CD47 blocking agent comprises soluble SIRPα having one or more conservative amino acid substitutions.
E17. The method, use, or drug-for-use according to any one of embodiments 15-16, wherein the CD47 blocking agent comprises an Fc fusion protein comprising the V region of soluble human SIRPα variant 2 attached to an antibody constant region (Fc).
E18. The method, use, or drug-for-use according to any one of embodiments 1-17, wherein the CD47⁺ cancer cells are blood cancer cells or solid tumour cells.
E19. The method, use, or drug-for-use according to embodiment 18, wherein the cancer cells are cells of a cancer type selected from acute lymphocytic leukemia (ALL); acute myeloid leukemia (AML) and p53 mutated AML; chronic lymphocytic leukemia (CLL); chronic myelogenous leukemia (CML); myeloproliferative disorder/neoplasm (MPDS); and myelodysplastic syndrome.
E20. The method, use, or drug-for-use according to embodiment 18, wherein the cancer cells are from a lymphoma selected from a T cell lymphoma, Hodgkin's lymphoma, indolent non-Hodgkin's lymphoma, aggressive non-Hodgkin's lymphoma, Burkitt's lymphoma, and small cell follicular lymphoma, and large cell follicular lymphoma.
E21. The method, use, or drug-for-use according to embodiment 18, wherein the cancer cells are from a myeloma selected from multiple myeloma (MM), giant cell myeloma, heavy-chain myeloma, and light chain or Bence-Jones myeloma.
E22. The method according to any one of embodiments 1-3 and 8-21, further comprising administering folic acid or Vitamin B12 to the subject.
E23. The use or drug-for-use according to any one of embodiments 4-21, for use in combination with folic acid or Vitamin B12.
E24. A combination of anti-cancer drugs, comprising an amount of a CD47 blocking agent effective to deplete CD47⁺ disease cells, and an amount of pralatrexate effective to enhance depletion of CD47⁺ disease cells, together with instructions teaching the use thereof according to any one of embodiments 1-23.
E25. The combination according to embodiment 24, for use in the treatment of a subject presenting with CD47⁺ disease cells.
E26. The combination-for-use according to embodiment 25, wherein the CD47⁺ disease cells are CD47⁺ cancer cells.
E27. The combination-for-use according to embodiment 26, wherein the CD47⁺ cancer cells comprise cells from a blood cancer or from a solid tumours.
E28. A kit comprising unit dose formulations of a CD47 blocking agent and a dihydrofolate reductase inhibitor (DHFRi).
   These and other aspects of the invention are now described in greater detail with reference to the accompanying drawings, in which:

### BRIEF DESCRIPTION OF THE DRAWINGS

FIG. 1 shows results from a macrophage phagocytosis assay on human lymphoma cell line HH. The bars show percentage phagocytosis for the following experimental conditions, from left to right: no treatment (“no Tx”); pralatrexate only (“Pra”); TTI-621 (SIRPα-IgG1 Fc)(“621”) only; combination of pralatrexate and TTI-621 (“Pra+621”).

FIG. 2 shows results from a macrophage phagocytosis assay on human lymphoma cell line H9. The bars show percentage phagocytosis for the following experimental conditions, from left to right: no treatment (“no Tx”); pralatrexate only (“Pra”); TTI-621 (SIRPα-IgG1 Fc)(“621”) only; combination of pralatrexate and TTI-621 (“Pra+621”).

FIG. 3 shows results from the macrophage phagocytosis assay of FIG. 1 (HH cells), in a different format, wherein the bars show the percentage phagocytosis greater than the no treatment condition (i.e. with the no treatment condition value subtracted). The conditions are, from left to right, pralatrexate only (“Pra”); TTI-621 (SIRPα-IgG1 Fc)(“621”) only; combination of pralatrexate and TTI-621 (“Pra+621”).

FIG. 4 shows results from the macrophage phagocytosis assay of FIG. 2 (H9 cells), in a different format, wherein the bars show the percentage phagocytosis greater than the no treatment condition (i.e. with the no treatment condition value subtracted). The conditions are, from left to right, pralatrexate only (“Pra”); TTI-621 (SIRPα-IgG1 Fc)(“621”) only; combination of pralatrexate and TTI-621 (“Pra+621”).

### DETAILED DESCRIPTION

The present invention provides an improved method for treating subjects that present with cancer cells and tumours that have a CD47+ phenotype. In this method, subjects receive a combination of a CD47 blockade drug (i.e., an anti-CD47 agent such as SIRPaFc) which can be any CD47-binding form of SIRPα that blocks signalling across the CD47/SIRPα axis, and a DHFRi. In combination, the anti-cancer effect of this combination is superior to the effects of either agent alone or of both agents in addition. The synergism is believed to result particularly when the CD47 blockade drug is a soluble SIRPα-based agent.

Thus, the present treatment method combines a CD47-binding and blocking form of SIRPα, as a CD47 blockade drug or blocking agent, and a DHFRi. An agent or drug that has CD47 blockade activity is an agent that interferes with and dampens signal transmission that results when CD47 interacts with macrophage-presented SIRPα. CD47-binding forms of human SIRPα are the preferred CD47 blockade drugs for use in the combination herein disclosed. These drugs are based on the extracellular region of human SIRPα. They comprise at least a region of the extracellular region sufficient to confer effective CD47 binding affinity and specificity. So-called “soluble” forms of SIRPα, lacking the membrane anchoring component, are described in the literature and include those referenced in Novartis WO 2010/070047, and Stanford's WO2013/109752, and Trillium's WO2014/094122, each incorporated by reference in its entirety.

In a preferred embodiment, the soluble form of SIRPα is an Fc fusion. More particularly, the drug suitably comprises the human SIRPα protein, in a form fused directly, or indirectly, with an antibody constant region, or Fc (fragment crystallisable). Unless otherwise stated, the term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of triggering signal transduction through CD47 upon binding thereto. The present invention is directed most particularly to the drug combinations that include the human SIRP variant 2 form, or V2.

In the present drug combination, useful SIRPαFc fusion proteins comprise one of the three so-called immunoglobulin (Ig) domains that lie within the extracellular region of human SIRPα. More particularly, the present SIRPαFc proteins incorporate residues 32-137 of human SIRPα (a 106-mer), which constitute and define the IgV domain of the V2 form according to current nomenclature. This SIRPα sequence, shown below, is referenced herein as SEQ ID NO: 1.

[SEQ ID NO: 1]
EELQVIQPDKSVSVAAGESAILHCTVTSLIPVGPIQWERGAGPARELIYN
QKEGHFPRVTTVSESTKRENMDFSISISNITPADAGTYYCVKFRKGSPDT
EFKSGA

In a preferred embodiment, the SIRPαFc fusion proteins incorporate the IgV domain as defined by SEQ ID NO: 1, and additional, flanking residues contiguous within the SIRPα sequence. This preferred form of the IgV domain, represented by residues 31-148 of the V2 form of human SIRPα, is a 118-mer having SEQ ID NO: 6 shown below:

[SEQ ID NO: 6]
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPS

The present SIRPα fusion proteins can also incorporate an Fc region having effector function. Fc refers to “fragment crystallisable” and represents the constant region of an antibody comprised principally of the heavy chain constant region and components within the hinge region. Suitable Fc components thus are those having effector function. An Fc component “having effector function” is an Fc component having at least some effector function, such as at least some contribution to antibody-dependent cellular cytotoxicity or some ability to fix complement. Also, the Fc will at least bind to Fc receptors. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Val234, Ala235 and deletion of Gly236 (EU), is considered not to have effector function. In a preferred embodiment, the Fc is based on human antibodies of the IgG1 isotype. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID NO: 2:

[SEQ ID NO: 2]
DKTHTCPPCPAPELLGGPSVFLFPPKPKDTLMISRTPEVTCVVVDVSHED
PEVKENWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK*

Thus, in embodiments, the Fc region has either a wild type or consensus sequence of an IgG1 constant region. In alternative embodiments, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469).

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is significantly less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In a specific embodiment, the Fc region is based on the amino acid sequence of a human IgG4 set out as P01861 in UniProtKB/Swiss-Prot, residues 99-327, and has the amino acid sequence shown below and referenced herein as SEQ ID NO: 7:

[SEQ ID NO: 7]
ESKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

In embodiments, the Fc region incorporates one or more alterations, usually not more than about 10, e.g., up to 5 such alterations, including amino acid substitutions that affect certain Fc properties. In one specific and preferred embodiment, the Fc region incorporates an alteration at position 228 (EU numbering), in which the serine at this position is substituted by a proline (S²²⁸P), thereby to stabilize the disulfide linkage within the Fc dimer. Other alterations within the Fc region can include substitutions that alter glycosylation, such as substitution of Asn²⁹⁷ by glycine or alanine; half-life enhancing alterations such as T²⁵²L, T²⁵³S, and T²⁵⁶F as taught in U.S. Pat. No. 62,777,375, and many others. Particularly useful are those alterations that enhance Fc properties while remaining silent with respect to conformation, e.g., retaining Fc receptor binding. In another embodiment, the Fc region is modified to increase its biological half-life. Various approaches are possible. For example, one or more of the following mutations can be introduced: T252L, T254S, T256F, as described in U.S. Pat. No. 6,277,375.

In a specific embodiment, and in the case where the Fc component is an IgG4 Fc, the Fc incorporates at least the S²²⁸P mutation, and has the amino acid sequence set out below and referenced herein as SEQ ID NO: 8:

[SEQ ID NO: 8]
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK

The CD47 blockade drug used in the combination is thus preferably a SIRP fusion protein useful to inhibit the binding of human SIRPα and human CD47, thereby to inhibit or reduce transmission of the signal mediated via SIRPα-bound CD47, the fusion protein comprising a human SIRPα component and, fused therewith, an Fc component, wherein the SIRPα component comprises or consists of a single IgV domain of human SIRPα V2 and the Fc component is the constant region of a human IgG having effector function.

In one embodiment, the fusion protein comprises a SIRPα component consisting at least of residues 32-137 of the V2 form of wild type human SIRPα, i.e., SEQ ID NO: 1. In a preferred embodiment, the SIRPα component consists of residues 31-148 of the V2 form of human SIRPα, i.e., SEQ ID NO: 6. In another embodiment, the Fc component is the Fc component of the human IgG1 designated P01857, and in a specific embodiment has the amino acid sequence that incorporates the lower hinge-CH2-CH3 region thereof i.e., SEQ ID NO: 2.

In a preferred embodiment, therefore, the SIRPαFc fusion protein is provided and used in a secreted dimeric fusion form, wherein the fusion protein incorporates a SIRPα component having SEQ ID NO: 1 and preferably SEQ ID NO: 6 and, fused therewith, an Fc region having effector function and having SEQ ID NO: 2. When the SIRPα component is SEQ ID NO: 1, this fusion protein comprises SEQ ID NO: 3, shown below:

[SEQ ID NO: 3]
EELQVIQPDKSVSVAAGESAILHCTVTSLIPVGPIQWFRGAGPARELIYN
QKEGHFPRVTTVSESTKRENMDFSISISNITPADAGTYYCVKFRKGSPDT
EFKSGAGTELSVRAKPSDKTHTCPPCPAPELLGGPSVFLFPPKPKDTLMI
SRTPEVTCVVVDVSHEDPEVKENWYVDGVEVHNAKTKPREEQYNSTYRVV
SVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK*

When the SIRPα component is SEQ ID NO: 6, this fusion protein comprises SEQ ID NO: 9, shown below:

[SEQ ID NO: 9]
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSDKTHTCPPCPAPELLGGPSVFLFPPKPKDTLM
ISRTPEVTCVVVDVSHEDPEVKENWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK

In alternative embodiments, the Fc component of the fusion protein is based on an IgG4, and preferably an IgG4 that incorporates the S²²⁸P mutation. In the case where the fusion protein incorporates the preferred SIRPα IgV domain of SEQ ID NO: 6, the resulting IgG4-based SIRPα-Fc protein has SEQ ID NO: 10, shown below:

[SEQ ID NO: 10]
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVESCSVMHEALHNHYTQKSLSLSLGK

In preferred embodiment, the fusion protein comprises, as the SIRPα IgV domain of the fusion protein, a sequence that is SEQ ID NO: 6. The preferred SIRPαFc is SEQ ID NO: 9 or SEQ ID NO: 10.

The SIRPα sequence incorporated within the CD47 blockade drug can be varied, as described in the literature. This can eliminate glycosylation sites in the protein, such as at position 89 and elsewhere. Other, useful substitutions within SIRPα include one or more of the following: L4V/I, V6I/L, A21V, V27I/L, 131T/S/F, E47V/L, K53R, E54Q, H56P/R, S66T/G, K68R, V92I, F94V/L, V63I, and/or F103V.

In the SIRPαFc fusion protein, the SIRPα component and the Fc component are fused, either directly or indirectly, to provide a single chain polypeptide that may optionally be ultimately produced as a dimer in which the single chain polypeptides are coupled through inter-chain disulfide bonds formed within the Fc region. The nature of the fusing region is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site, such as a BamHI, ClaI, EcoRI, HindIII, PstI, SalI and XhoI site and the like.

The linker amino acids typically and desirably have some flexibility to allow the Fc and the SIRP components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G4S sequence (Gly-Gly-Gly-Gly-Ser [SEQ ID NO: 5]) which may repeat as (G4S)n where n is 1, 2, 3 or more, or is based on (Gly)n, (Ser)n, (Ser-Gly)n or (Gly-Ser)n and the like. In another embodiment, the linker is GTELSVRAKPS [SEQ ID NO: 4]. This sequence constitutes SIRPα sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

As noted, the SIRPαFc fusion is useful to inhibit interaction between SIRPα and CD47, thereby to block signalling across this axis. Stimulation of SIRPα on macrophages by CD47 is known to inhibit macrophage-mediated phagocytosis by deactivating myosin-II and the contractile cytoskeletal activity involved in pulling a target into a macrophage. Activation of this cascade is therefore important for the survival of CD47⁺ disease cells, and blocking this pathway enables macrophages to eradicate or at least reduce the CD47⁺ disease cell population. A CD47 blockade drug thus can be any agent that achieves this end, including a CD47 antibody and bispecific forms thereof, as well as a CD47Fc fusion or a SIRPα antibody.

The term “CD47⁺” (or CD47+) is used with reference to the phenotype of cells targeted for binding by the present polypeptides. Cells that are CD47⁺ can be identified by flow cytometry using CD47 antibody as the affinity ligand. CD47 antibodies that are labeled appropriately are available commercially for this use (for example, the antibody product of clone B6H12 is available from Santa Cruz Biotechnology). The cells examined for CD47 phenotype can include standard tumour biopsy samples including particularly blood samples taken from the subject suspected of harbouring endogenous CD47⁺ cancer cells. CD47 disease cells of particular interest as targets for therapy with the present fusion proteins are those that “over-express” CD47. These CD47⁺ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry as exemplified herein or by immunostaining or by gene expression analysis or the like, to be greater than the level measurable on a counterpart cell having a CD47 phenotype that is normal for that cell type.

The present drug combination comprises both a CD47 blocking agent that is a CD47-binding form of a SIRPα, as just described, and a DHFRi. In a preferred embodiment, the DHFRi is pralatrexate and the CD47 blocking agent is a CD47-binding form of SIRPaFc

Pralatrexate is sold currently under the name Folotyn® (Acrotech Biopharma). It is a medication used for the treatment of various cancers, including but not limited to relapsed or refractory peripheral T cell lymphoma, an often-aggressive form of non-Hodgkin's Lymphoma. It has the structure shown below:

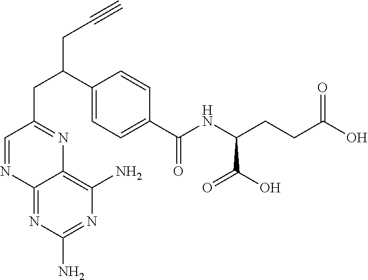 [SMILES: C#CCC(Cc1cnc2nc(N)nc(N)c2n1)c1ccc(C(=O)N[C@@H](CCC(=O)O)C(=O)O)cc1]

Pralatrexate is given by intravenous (IV) injection. Folic acid and vitamin B12 supplements are also prescribed during treatment with pralatrexate to reduce the risk of possible side effects. Pralatrexate exerts its chemotherapeutic effect by being able to counteract and compete with folic acid in cancer cells resulting in folic acid deficiency in the cells and causing their death.

Other forms of anti-folates (folate derivatives) that can also be used in combination with the SIRPaFc or other CD47 blocking agent (an agent that blocks binding between SIRP and CD47), include methotrexate, raltitrexed, and pemetrexed and these are embodiments of the present invention.

Each drug included in the combination can be formulated separately for use in combination. The drugs are said to be used “in combination” and to produce a desired effect or to comprise effective amounts, when, in a recipient of both drugs, the effect of one drug enhances the effect of the other.

In this approach, each drug is provided in a dosage form comprising a pharmaceutically acceptable carrier, and in a therapeutically effective amount. As used herein, “pharmaceutically acceptable carrier” means any and all solvents, dispersion media, coatings, antibacterial and antifungal agents, isotonic and absorption delaying agents, and the like that are physiologically compatible and useful in the art of protein/antibody formulation. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent. The SIRPαFc fusion protein is formulated using practices standard in the art of therapeutic protein formulation. Solutions such as saline that are suitable for intravenous administration, such as by injection or infusion, are particularly useful. The DHFRi will be formulated as permitted by the regulatory agencies that have approved its use in humans.

Sterile solutions can be prepared by incorporating the active compound in the required amount in an appropriate solvent with one or a combination of ingredients noted above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation are vacuum drying and freeze-drying (lyophilization) that yield a powder of the active ingredient plus any additional desired ingredient from a previously sterile-filtered solution thereof.

As used herein, “effective amount” refers to an amount effective, at dosages and for a particular period of time necessary, to achieve the desired therapeutic result. A therapeutically effective amount of each drug in the combination may vary according to factors such as the disease state, age, sex, and weight of the individual, and the ability of the drug to elicit a desired response in the recipient. A therapeutically effective amount is also one in which any toxic or detrimental effects of the pharmacological agent are outweighed by the therapeutically beneficial effects. The DHFRi will be formulated in amounts that are suitable for patient dosing, as permitted by the regulatory agencies that have approved its use in humans. For pralatrexate, effective doses will include 30 mg/m2 via intravenous push over 3 to 5 minutes once weekly for 6 weeks in 7 week cycles, until disease progression or unacceptable toxicity. For SIRPaFc, TTI-621, exemplary dosing would be between 0.2-2.0 mg/kg IV weekly, or possibly less frequent (Q2W or Q3W) administration.

Patients treated with the present combination may, because of pralatrexate dosing, take low dose (1 mg to 1.25 mg) oral folic acid daily. Folic acid may start 10 days before the first dose of pralatrexate and continue for 30 days after the last dose. Patients may also receive a B12 (1 mg) injection within 10 weeks before the first dose of pralatrexate and every 8 to 10 weeks thereafter. Subsequent B12 injections may be given the same day as treatment with pralatrexate. 2-4 milligrams given intravenously such as by infusion over the course of 5-15 minutes, for instance.

The SIRPαFc fusion protein can be administered to the subject through any of the routes established for protein delivery, in particular intravenous, intradermal and subcutaneous injection or infusion, or by oral or nasal administration.

The drugs in the present combination can be administered sequentially or, essentially at the same time. In embodiments, the DHFRi is given before administration of SIRPαFc. It is not essential that the DHFRi is present in a patient's system when the CD47 blockade drug is administered, although this is suitable. Thus, in one embodiment there is provided a method for treating a subject presenting with CD47⁺ disease cells, comprising administering pralatrexate to the subject and then administering SIRPαFc to that subject in amounts sufficient to reduce the disease cell population.

Dosing regimens are adjusted to provide the optimum desired response (e.g., a therapeutic response). For example, a single bolus of each drug may be administered, or several divided doses may be administered over time or the dose may be proportionally reduced or increased as indicated by the therapeutic situation. It is especially advantageous to formulate parenteral compositions in unit dosage form for ease of administration and uniformity of dosage. “Unit dosage form” as used herein refers to physically discrete units suited as unitary dosages for the subjects to be treated; each unit contains a predetermined quantity of active compound calculated to produce the desired therapeutic effect in association with the required pharmaceutical carrier.

The drugs can be formulated in combination, so that the combination can be introduced to the recipient in one administration, e.g., one injection or one infusion bag. Alternatively, the drugs can be combined as separate units that are provided together in a single package, and with instructions for the use thereof according to the present method. In another embodiment, an article of manufacture containing the SIRPαFc drug and DHFRi combination in an amount useful for the treatment of the disorders described herein is provided. The article of manufacture comprises one or both drugs of the present antibody drug combination, as well as a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or vial having a stopper pierceable by a hypodermic injection needle). The label on or associated with the container indicates that the composition is used in combination with another CD47 blockade drug in accordance with the present invention, thereby to elicit a synergistic effect on the CD47⁺ disease cells. The article of manufacture may further comprise a second container comprising a pharmaceutically-acceptable buffer, such as phosphate-buffered saline, Ringer's solution and dextrose solution. It may further include other matters desirable from a commercial and use standpoint, including other buffers, diluents, filters, needles, syringes, and package inserts with instructions for use.

For administration the dose for the CD47 blockade drug will be within the range from about 0.0001 to 100 mg/kg, when TTI-621 is used and more usually 0.01 to 30 mg/kg, of the host body weight. For example, dosages can be 0.3 mg/kg body weight, 1 mg/kg body weight, 3 mg/kg body weight, 5 mg/kg body weight or 10 mg/kg body weight. Higher doses can be used when the drug is TTI-622 (SEQ ID NO: 10) (SIRPaFc where the Fc is a G4 isotype and a substitution occurs in the Fc as S²²⁸P), such as within the general range of 0.1-50 mg/kg.

The SIRPαFc protein displays negligible binding to red blood cells. There is accordingly no need to account for an RBC “sink” when dosing with the drug combination. Relative to other CD47 blockade drugs that are bound by RBCs, it is estimated that the present SIRPαFc fusion can be effective at doses that are less than half the doses required for drugs that become RBC-bound, such as CD47 antibodies. Moreover, the SIRPα-Fc fusion protein is a dedicated antagonist of the SIRPα-mediated signal, as it displays negligible CD47 agonism when binding thereto. There is accordingly no need, when establishing medically useful unit dosing regimens, to account for any stimulation induced by the drug.

The drug combination is useful to treat a variety of CD47⁺ disease cells. These include particularly CD47⁺ cancer cells, including liquid (hematological) and solid tumours. The anti-folates (DHFRi) themselves, and thus the combinations also, are used for treatment of leukemia lymphoma, osteosarcoma, non-small cell lung cancer, mesothelioma, colorectal cancer and breast cancer. Solid tumours can be treated with the present drug combination, to reduce the size, number or growth rate thereof and to control growth of cancer stem cells. Such solid tumours include CD47⁺ tumours in bladder, brain, breast, lung, colon, ovary, prostate, liver and other tissues as well. In one embodiment, the drug combination can used to inhibit the growth or proliferation of hematological cancers. As used herein, “hematological cancer” refers to a cancer of the blood, and includes leukemia, lymphoma and myeloma among others. “Leukemia” refers to a cancer of the blood, in which too many white blood cells that are ineffective in fighting infection are made, thus crowding out the other parts that make up the blood, such as platelets and red blood cells. It is understood that cases of leukemia are classified as acute or chronic. Certain forms of leukemia may be, by way of example, acute lymphocytic leukemia (ALL); acute myeloid leukemia (AML); chronic lymphocytic leukemia (CLL); chronic myelogenous leukemia (CML); myeloproliferative disorder/neoplasm (MPDS); and myelodysplastic syndrome. “Lymphoma” may refer to a Hodgkin's lymphoma, both indolent and aggressive non-Hodgkin's lymphoma, Burkitt's lymphoma, and follicular lymphoma (small cell and large cell), among others. Myeloma may refer to multiple myeloma (MM), giant cell myeloma, heavy-chain myeloma, and light chain or Bence-Jones myeloma. In particular embodiments, the combination is useful to treat T cell lymphomas that are a very heterogeneous group of lymphoid malignancies divided into cutaneous and peripheral TCL, which themselves are divided into nodal or extranodal types. CTCL derive from skin-homing T cells and consist of mycosis fungoides, Sezary syndrome, primary cutaneous T cell lymphoproliferative disorders, and anaplastic large cell lymphoma. The common features of TCL are aggressive course and poor response to therapy, with the exception of ALK and ALCL.

In some other embodiments, the hematological cancer treated with the drug combination is a CD47⁺ leukemia, preferably selected from acute lymphocytic leukemia, acute myeloid leukemia, chronic lymphocytic leukemia, chronic myelogenous leukemia, and myelodysplastic syndrome, preferably, human acute myeloid leukemia.

In other embodiments, the hematological cancer treated with the drug combination is a CD47⁺ lymphoma or myeloma selected from Hodgkin's lymphoma, both indolent and aggressive non-Hodgkin's lymphoma, Burkitt's lymphoma, follicular lymphoma (small cell and large cell), multiple myeloma (MM), giant cell myeloma, heavy-chain myeloma, and light chain or Bence-Jones myeloma as well as leiomyosarcoma.

In human non-small cell lung cancer (NSCLC) xenograft, pralatrexate showed increased antitumor activity. In the 2 mg/kg pralatrexate-treated group, 38% tumor growth inhibition (TGI) was observed. In NCI-H460 NSCLC xenograft, pralatrexate showed antitumor activity in a dose-dependent way. The TGI of 1 mg/kg and 2 mg/kg pralatrexate-treated groups was 34% and 52%, respectively. Thus, the present combination can be useful to treat solid tumours such as lung tumours and tumours of other solid tissues.

The combination therapy, comprising CD47 blockade and anti-folate such as pralatrexate, can also be exploited together with any other agent or modality useful in the treatment of the targeted indication, such as surgery as in adjuvant therapy, or with additional chemotherapy as in neoadjuvant therapy.

### Examples

A macrophage phagocytosis assay was conducted in order to assess the effects of the combination of a DHFRi and CD47 blocking agent as compared to the agents individually on macrophage phagocytosis of human lymphoma cells.

For the assay, the DHFRi was pralatrexate, and the CD47 blocking agent was an Fc fusion protein comprising soluble SIRPα (TTI-621). The lymphoma cells were human T cell lymphoma cell lines HH (ATCC Ref. CRL-2105) (a mature T cell line from peripheral blood of a patient with aggressive cutaneous T cell leukemia/lymphoma) or H9 (ATCC Ref. HTB-176) (a cutaneous T cell lymphoma). Thus, there were 4 experimental conditions for each lymphoma cell type: A) no treatment; B) pralatrexate only; C) TTI-621 (SIRPα-IgG1 Fc) only; and D) combination of pralatrexate and TTI-621.

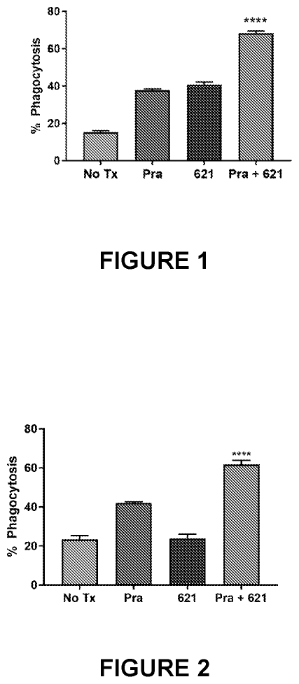

PBMC from normal donors were purchased from BioIVT and informed consent was obtained from all donors. CD14+ monocytes were isolated from PBMCs by positive selection using human monocyte isolation kit. Monocytes were differentiated into macrophages by culturing for at least ten days in X-Vivo-15 media (Lonza) supplemented with M-CSF (PeproTech), at which point, for the pralatrexate treatment experimental conditions, pralatrexate (Selleckchem) was added to the macrophage culture for an additional three days. Similarly, for the pralatrexate treatment experimental conditions, human lymphoma cell lines HH or H9 were also treated with pralatrexate (Selleckchem) for three days prior to the phagocytosis assay. One day before the phagocytosis assay, macrophages were primed with IFNg (PeproTech). On the day of the phagocytosis assay, macrophages were co-cultured with violet proliferation dye 450 (VPD450)-treated HH or H9 cells for two hours and, for the TTI-621 treatment conditions, TTI-621 was added prior to the two hour co-culture. Phagocytosis was assessed as % VPD450+ cells of live, single CD14+CD11b+ macrophages by flow cytometry. Results are shown in FIGS. 1-4.

FIG. 1 shows macrophage phagocytosis of cell line HH and ****p<0.0001 for the combination treatment of pralatrexate+TTI-621 vs. single agents alone or no-treatment control established by one-way ANOVA. FIG. 2 shows macrophage phagocytosis of cell line H9 and ****p<0.0001 for the combination treatment of pralatrexate+TTI-621 vs. single agents alone or no treatment control established by one-way ANOVA. As shown in FIGS. 1 and 2, when macrophages cultured with cancer cells were treated with the combination of pralatrexate and TTI-621 (SEQ ID NO: 9), there was a significant increase in cancer cell phagocytosis versus treatment with single agents alone, or no-treatment control. In addition, this effect was present for both a CD47-sensitive cell line (HH cells; FIG. 1) and a CD47-insensitive cell line (H9 cells; FIG. 2). (For indication of CD47-sensitivity, compare % phagocytosis for 621 alone for HH cells vs H9 cells; there is increased phagocytosis of HH cells with 621 alone as compared to the no-treatment control; this increase with 621 alone is not present for the H9 cells.)

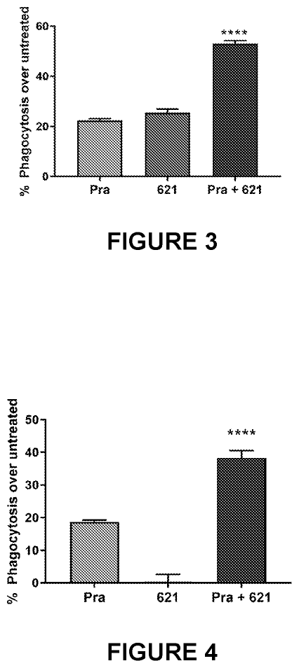

The data in FIGS. 1 and 2 was then re-evaluated with background subtracted; this is provided in FIGS. 3 and 4, respectively. As shown in FIGS. 3 and 4, the percentage phagocytosis of the combination is greater than the added percentage phagocytosis from SIRPaFc and pralatrexate separately, for both HH cells (FIG. 3) and H9 cells (FIG. 4). Thus, there are significant and even synergistic effects for the combination of pralatrexate and TTI-621 as compared to the single agent treatment.

All patent and patent publications referenced herein are incorporated by reference in their entirety. All sequence database entries referenced herein, including but not limited to entries in UniProtKB/Swiss-Prot and/or GenBank, are incorporated herein with respect to versions in existence as of Apr. 27, 2021.

Incorporated by reference herein for all purposes is the content of U.S. Provisional Patent Application Nos. 63/180,604 (filed Apr. 27, 2021) and 63/253,125 (filed Oct. 6, 2021).

1. A method for treating a subject presenting with CD47⁺ cancer cells, comprising administering to the subject a CD47 blocking agent, and a dihydrofolate reductase inhibitor (DHFRi).

2-7. (canceled)

8. The method according to claim 1, wherein the DHFRi comprises methotrexate or pralatrexate.

9. The method according to claim 1, wherein the DHFRi comprises pralatrexate.

10. The method according to claim 1, wherein the CD47 blocking agent comprises a CD47-binding form of human SIRPα.

11. The method according to claim 10, wherein the CD47-binding form of human SIRPα is a CD47-binding fragment of human SIRPα.

12. The method according to claim 11, wherein the CD47 binding fragment of human SIRPα comprises the V region of human SIRPα.

13. The method according to claim 1, wherein the CD47 blocking agent comprises an Fc fusion protein comprising the V region of soluble human SIRPα variant 2 attached to an antibody constant region (Fc).

14. The method according to claim 13, wherein the Fc fusion protein comprising soluble SIRPα comprises SEQ ID NO: 9 or SEQ ID NO: 10.

15-17. (canceled)

18. The method according to claim 1, wherein the CD47⁺ cancer cells are blood cancer cells or solid tumour cells.

19. The method according to claim 18, wherein the cancer cells are cells of a cancer type selected from acute lymphocytic leukemia (ALL); acute myeloid leukemia (AML) and p53 mutated AML; chronic lymphocytic leukemia (CLL); chronic myelogenous leukemia (CML); myeloproliferative disorder/neoplasm (MPDS); and myelodysplastic syndrome.

20. The method according to claim 18, wherein the cancer cells are from a lymphoma selected from a T cell lymphoma, Hodgkin's lymphoma, indolent non-Hodgkin's lymphoma, aggressive non-Hodgkin's lymphoma, Burkitt's lymphoma, and small cell follicular lymphoma, and large cell follicular lymphoma.

21. The method according to claim 18, wherein the cancer cells are from a myeloma selected from multiple myeloma (MM), giant cell myeloma, heavy-chain myeloma, and light chain or Bence-Jones myeloma.

22. The method according to claim 1, further comprising administering folic acid or Vitamin B12 to the subject.

23. (canceled)

24. A combination of anti-cancer drugs, comprising an amount of a CD47 blocking agent effective to deplete CD47⁺ disease cells, and an amount of pralatrexate effective to enhance depletion of CD47⁺ disease cells, together with instructions teaching the use thereof according to claim 1.

25. The combination according to claim 24, for use in the treatment of a subject presenting with CD47⁺ disease cells.

26. The combination-for-use according to claim 25, wherein the CD47⁺ disease cells are CD47⁺ cancer cells.

27. The combination-for-use according to claim 26, wherein the CD47⁺ cancer cells comprise cells from a blood cancer or from a solid tumours.

28. A kit comprising unit dose formulations of a CD47 blocking agent and a dihydrofolate reductase inhibitor (DHFRi).


SEQUENCE LISTING (10 sequences)

SEQ ID NO: 1 - protein, 106 aa, Artificial Sequence
  Synthetic
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 2 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 344 aa, Artificial Sequence
  Synthetic
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 11 aa, Artificial Sequence
  Synthetic
GTELSVRAKPS

SEQ ID NO: 5 - protein, 5 aa, Artificial Sequence
  Synthetic
GGGGS

SEQ ID NO: 6 - protein, 118 aa, Artificial Sequence
  Synthetic
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 7 - protein, 229 aa, Artificial Sequence
  Synthetic
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 229 aa, Artificial Sequence
  Synthetic
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 345 aa, Artificial Sequence
  Synthetic
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 347 aa, Artificial Sequence
  Synthetic
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK